(19) United States
(12) Patent Application Publication
(10) Pub. No.: US 2022/0058971 A1
(43) Pub. Date: Feb. 24, 2022

(54) DYNAMIC TARGET RATE FOR GUIDED BREATHING

(71) Applicant: BOSE CORPORATION, Framingham, MA (US)

(72) Inventors: Harsh A. MANKODI, Brighton, MA (US); Michelle DANIELS, Framingham, MA (US); Sara ADKINS, Allston, MA (US); Chia-Chun HSU, Brighton, MA (US); Chia-Ling LI, Framingham, MA (US); Kathleen Elizabeth KREMER, Morris Plains, NJ (US)

(21) Appl. No.: 17/400,403

(22) Filed: Aug. 12, 2021

Related U.S. Application Data
(60) Provisional application No. 63/067,679, filed on Aug. 19, 2020.

(51) Int. Cl. G09B 5/00; A63B 23/18

(52) U.S. Cl. CPC G09B 5/00; G06F 3/165; A63B 23/185

(57) ABSTRACT

Aspects of the present disclosure provide methods, apparatuses, and systems for dynamically adjusting a breathing entrainment based on whether a user is stressing or relaxing. According to an aspect, a first target breathing period is selected, and a guiding stimulus configured to alter a current breathing period of the user towards the first target breathing period over an interval of time is output. One or more relaxation biometrics are measured and analyzed to determine whether the user is stressing or relaxing. Based on whether the user is relaxing or stressing, at least one of the guided stimulus and the first target breathing period are adjusted, where the first target breathing period is adjusted to a second target breathing period different from the first target breathing period. By making adjustments based on whether a user is relaxing or stressing, the breathing entrainment is more effective and comfortable for users.

### CROSS-REFERENCE TO RELATED APPLICATION

This application claims priority to and benefit of U.S. Provisional Patent Application No. 63/067,679, titled “DYNAMIC TARGET RATE FOR GUIDED BREATHING”, filed Aug. 19, 2020, the contents of which are herein incorporated by reference in its entirety as fully set forth below.

### FIELD

Aspects of the present disclosure generally relate to methods, apparatuses, and systems for breathing entrainment.

### BACKGROUND

Utilizing breathing entrainment to regulate a user or subject's breathing rate, or amount of breaths taken per minute, can be beneficial in a number of health fields. For example, breathing entrainment can be used in several clinical applications, potentially leading to more effective or quicker treatments of conditions, including: asthma, stress, anxiety, insomnia, panic disorder, recurrent abdominal pain, chronic obstructive pulmonary disease, chronic hyperventilation, hypertension, and congestive heart failure, among others. Breathing entrainment may also be utilized to assist people in falling asleep and for meditation or relaxation purposes.

However, many breathing entrainment exercises decrease a user's amount of breaths per minute in a linear or step-wise manner by reducing the amount of breaths taken per minute by one full breath. For instance, if a user follows the breathing entrainment sequence for one minute taking 9 breaths per minute, the next reduction is to 8 breaths per minute, and so on. This type of breathing entrainment sequence may be uncomfortable for some users, being too slow or too quick for a user to follow. As such, the breathing entrainment may cause a user stress, rather than assisting the user in relaxing. Therefore, there is a need for a breathing entrainment method that is able to take a user's stress and relaxation into account dynamically.

### SUMMARY

Aspects of the present disclosure provide methods, apparatuses, and systems for dynamically adjusting a breathing entrainment based on whether a user is stressing or relaxing. According to an aspect, a first target breathing period is selected, and a guiding stimulus configured to alter a current breathing period of the user towards the first target breathing period over an interval of time is output. One or more relaxation biometrics are measured and analyzed to determine whether the user is stressing or relaxing. Based on whether the user is relaxing or stressing, at least one of the guided stimulus and the first target breathing period are adjusted, where the first target breathing period is adjusted to a second target breathing period different from the first target breathing period. By making adjustments based on whether a user is relaxing or stressing, the breathing entrainment is more effective and comfortable for users.

In an aspect, a method for breathing entrainment comprises selecting a first target breathing period. A breathing period is an amount of time from a beginning of one inhale to a beginning of a next inhale. The method further comprises outputting a guiding stimulus. The guiding stimulus is configured to alter the current breathing period towards the final breathing period over an interval of time at a non-linear prescribed rate. The method further comprises determining one or more relaxation biometrics of the user that indicate whether the user is relaxing or stressing. Based at least in part on the one or more determined relaxation biometrics of the user, adjusting at least one of: the guiding stimulus, and the first target breathing period, the first target breathing period being adjusted to a second target breathing period.

Determining the one or more relaxation biometrics of the user comprises detecting a resonance frequency of the user. The resonance frequency is a respiratory rate of the user indicative of relaxation. Adjusting at least one of the guiding stimulus and the first target breathing period comprises performing at least one of: pausing or increasing a rate of decay of the non-linear prescribed rate of the guiding stimulus in response to detecting the resonance frequency of the user, adjusting a haptic stimulus rate of the guiding stimulus, adjusting a haptic stimulus intensity of the guiding stimulus, adjusting an audio output of the guided stimulus to sounds of higher complexity, adjusting a visual output of the guided stimulus, and adjusting a thermal output of the guided stimulus.

Adjusting at least one of the guiding stimulus and the first target breathing period comprises adjusting the first target breathing period to the second target breathing period in response to detecting the resonance frequency of the user. The second target breathing period is at least one of: the current breathing period of the user at the time the resonance frequency is detected, or a lower breathing period than the first target breathing period.

Determining the one or more relaxation biometrics of the user comprises determining the user is stressed based on the one or more relaxation biometrics of the user.

Adjusting at least one of the guiding stimulus and the first target breathing period comprises performing at least one of: decreasing a volume of an audio output of the guiding stimulus, adjusting the audio output of the guiding stimulus to sounds of higher soothing quality, adjusting the audio output of the guiding stimulus to sounds of lower complexity, adjusting the first target breathing period to the second target breathing period, the second target breathing period having a higher breathing rate than the first target breathing period, reducing a rate of decay of the non-linear prescribed rate of the guiding stimulus, reducing a haptic stimulus intensity of the guiding stimulus, adjusting a haptic stimulus rate of the guiding stimulus, adjusting a visual output of the guided stimulus, and adjusting a thermal output of the guided stimulus.

The one or more relaxation biometrics are determined using one or more biometric sensors. The guiding stimulus is at least one of audio output, a haptic stimulus, a visual output, and a thermal output. The one or more relaxation biometrics are selected from the group consisting of heart rate, heart rate variability, electrodermal activity, electromyography, respiration, perspiration, and blood pressure.

In another aspect, a wearable audio device comprises at least one biosensor for determining one or more relaxation biometrics of a user that indicate whether the user is relaxing or stressing. The wearable audio device further comprises at least one speaker configured to output a guiding stimulus. The guiding stimulus is configured to alter a current breathing period of the user towards a first target breathing period over an interval of time at a non-linear prescribed rate. A breathing period is an amount of time from a beginning of one inhale to a beginning of a next inhale. The wearable audio device further comprises a processing unit or processor configured to select the first target breathing period, and based at least in part on the one or more determined relaxation biometrics of the user, adjust at least one of: the guiding stimulus, and the first target breathing period, the first target breathing period being adjusted to a second target breathing period.

Determining the one or more relaxation biometrics of the user comprises detecting a resonance frequency of the user. The resonance frequency is a respiratory rate of the user indicative of relaxation.

Adjusting at least one of the guiding stimulus and the first target breathing period comprises performing at least one of: pausing or increasing a rate of decay of the non-linear prescribed rate of the guiding stimulus in response to detecting the resonance frequency of the user, adjusting a haptic stimulus rate of the guiding stimulus, adjusting a haptic stimulus intensity of the guiding stimulus, adjusting an audio output of the guided stimulus to sounds of higher complexity, adjusting a visual output of the guided stimulus, and adjusting a thermal output of the guided stimulus.

Adjusting at least one of the guiding stimulus and the first target breathing period comprises adjusting the first target breathing period to the second target breathing period in response to detecting the resonance frequency of the user. The second target breathing period is at least one of: the current breathing period of the user at the time the resonance frequency is detected, or a lower breathing period than the first target breathing period.

Determining the one or more relaxation biometrics of the user comprises determining the user is stressed based on the one or more relaxation biometrics of the user. Adjusting at least one of the guiding stimulus and the first target breathing period comprises performing at least one of: decreasing a volume of an audio output of the guiding stimulus, adjusting the audio output of the guiding stimulus to sounds of higher soothing quality, adjusting the audio output of the guiding stimulus to sounds of lower complexity, adjusting the first target breathing period to the second target breathing period, the second target breathing period having a higher breathing rate than the first target breathing period, reducing a rate of decay of the non-linear prescribed rate of the guiding stimulus, reducing a haptic stimulus intensity of the guiding stimulus, adjusting a haptic stimulus rate of the guiding stimulus, adjusting a visual output of the guided stimulus, and adjusting a thermal output of the guided stimulus.

The one or more relaxation biometrics are determined using one or more biometric sensors. The guiding stimulus is at least one of audio output, a haptic stimulus, a visual output, and a thermal output. The one or more relaxation biometrics are selected from the group consisting of heart rate, heart rate variability, electrodermal activity, electromyography, respiration, perspiration, and blood pressure.

In yet another aspect, an audio system comprises at least one biosensor for determining one or more relaxation biometrics of a user that indicate whether the user is relaxing or stressing. The audio system device further comprises at least one speaker configured to output a guiding stimulus. The guiding stimulus is configured to alter a current breathing period of the user towards a first target breathing period over an interval of time at a non-linear prescribed rate. A breathing period is an amount of time from a beginning of one inhale to a beginning of a next inhale. The audio system further comprises a processing unit or processor configured to select the first target breathing period, and based at least in part on the one or more determined relaxation biometrics of the user, adjust at least one of: the guiding stimulus, and the first target breathing period, the first target breathing period being adjusted to a second target breathing period.

Determining the one or more relaxation biometrics of the user comprises detecting a resonance frequency of the user. The resonance frequency is a respiratory rate of the user indicative of relaxation.

Adjusting at least one of the guiding stimulus and the first target breathing period comprises performing at least one of: pausing or increasing a rate of decay of the non-linear prescribed rate of the guiding stimulus in response to detecting the resonance frequency of the user, adjusting a haptic stimulus rate of the guiding stimulus, adjusting a haptic stimulus intensity of the guiding stimulus, adjusting an audio output of the guided stimulus to sounds of higher complexity, adjusting a visual output of the guided stimulus, and adjusting a thermal output of the guided stimulus.

Adjusting at least one of the guiding stimulus and the first target breathing period comprises adjusting the first target breathing period to the second target breathing period in response to detecting the resonance frequency of the user. The second target breathing period is at least one of: the current breathing period of the user at the time the resonance frequency is detected, or a lower breathing period than the first target breathing period.

Determining the one or more relaxation biometrics of the user comprises determining the user is stressed based on the one or more relaxation biometrics of the user. Adjusting at least one of the guiding stimulus and the first target breathing period comprises performing at least one of: decreasing a volume of an audio output of the guiding stimulus, adjusting the audio output of the guiding stimulus to sounds of higher soothing quality, adjusting the audio output of the guiding stimulus to sounds of lower complexity, adjusting the first target breathing period to the second target breathing period, the second target breathing period having a higher breathing rate than the first target breathing period, reducing a rate of decay of the non-linear prescribed rate of the guiding stimulus, reducing a haptic stimulus intensity of the guiding stimulus, adjusting a haptic stimulus rate of the guiding stimulus, adjusting a visual output of the guided stimulus, and adjusting a thermal output of the guided stimulus.

The one or more relaxation biometrics are determined using one or more biometric sensors. The guiding stimulus is at least one of audio output, a haptic stimulus, a visual output, and a thermal output. The one or more relaxation biometrics are selected from the group consisting of heart rate, heart rate variability, electrodermal activity, electromyography, respiration, perspiration, and blood pressure.

All examples and features mentioned herein can be combined in any technically possible manner.

### BRIEF DESCRIPTION OF THE DRAWINGS

FIG. 1 illustrates an example audio system in a sleeping environment.

FIG. 2 illustrates example components of an audio device.

FIG. 3A illustrates an example method for dynamically adjusting breathing entrainment based on a user's state of relaxation, according to one embodiment.

FIG. 3B illustrates an example method of dynamically adjusting breathing entrainment when the user is determined to be relaxing.

FIG. 3C illustrates an example method of dynamically adjusting breathing entrainment when the user is determined to be stressing.

### DETAILED DESCRIPTION

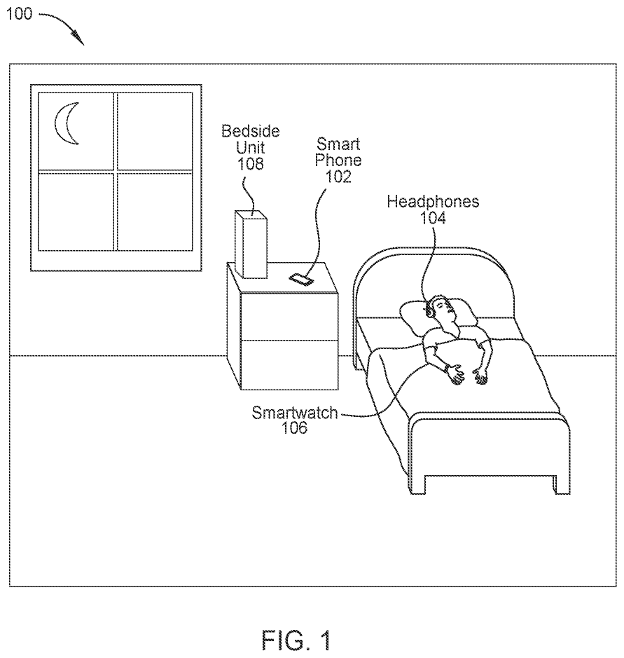

FIG. 1 illustrates an example audio system 100 in a sleeping environment, according to an aspect. The audio system 100 may be used to select a first target breathing period, a breathing period being an amount of time from a beginning of one inhale to a beginning of a next inhale, output a guiding stimulus to a user, determine one or more relaxation biometrics of the user, analyzing the one or more relaxation biometrics to determine whether the user is relaxing or stressing, and based at least in part on the one or more determined relaxation biometrics of the user, adjusting at least one of: the guiding stimulus, and the first target breathing period, the first target breathing period being adjusted to a second target breathing period.

The audio system 100 includes headphones 104 and a wearable sensor device 106, such as a smartwatch, which are shown as being worn by a subject or user. A headphone 104 refers to a device that fits around, on, or in an ear and that radiates acoustic energy into the ear canal. Headphones 104 are sometimes referred to as earphones, earpieces, headsets, earbuds, or sport headphones, and can be wired or wireless. The headphones 104 may comprise one or more of: a processing unit, a transceiver, one or more biosensors, one or more speakers, and one or more microphones. The headphones 104 may comprise an interface configured to receive input from a subject or user. A wearable sensor device 106 may be any type of wearable computer designed to be worn on a wrist of a subject or user, such as a fitness tracker, or any other type of wearable sensor device, such as a chest strap, a sleep mask, or a sensor integrated into the clothing or accessories of a user. The wearable sensor device 106 may comprise one or more of: a processing unit, a transceiver, one or more biosensors, one or more speakers, and one or more microphones. The wearable sensor device 106 may comprise an interface configured to receive input from a subject or user. The wearable sensor device may be configured to detect perspiration of a user.

The audio system 100 further includes a bedside unit 108 and a smartphone 102. The smartphone 102 may be a mobile phone, tablet, phablet, or laptop computer. The smartphone 102 may comprise one or more of: a processing unit, a transceiver, one or more biosensors, one or more speakers, and one or more microphones. The smartphone 102 may comprise an interface configured to receive input from a subject or user. The bedside unit 108 may be a stationary smart device, such as a smart speaker or an aromatherapy diffuser. The bedside unit 108 may have any shape and size capable of fitting on a surface in the sleeping environment, such as a dresser, desk, or night table. The bedside unit 108 may comprise one or more of: a processing unit, a transceiver, one or more biosensors, one or more speakers, and one or more microphones. In one embodiment, the bedside unit 108 comprises one or more contactless biosensors, such as a radio frequency (RF) sensor, a radar sensor, or an under-bed accelerometer. The bedside unit 108 may comprise an interface configured to receive input from a subject or user.

The one or more biosensors of the smart phone 102, the headphones 104, the wearable sensor device 106, and/or the bedside unit 108 may include a heart rate sensor, a wearable accelerometer, a wearable gyroscope, a wearable sweat sensor, a chest-strap, a RF sensor, a radar sensor, an under-bed accelerometer, an under-bed gyroscope, a motion sensor, among others. The one or more biosensors are configured to determine one or more relaxation biometrics of a user that indicate whether the user is relaxing or stressing. For example, the one or more relaxation biometrics may include heart rate, heart rate variability, electrodermal activity (EDA), electromyography (EMG), respiration, perspiration, and blood pressure, among others. As the user is close to falling asleep, it is natural for the body to have hypnic jerks which are captured with EMG. The one or more relaxation biometrics may be determined from one or more of the smart phone 102, the headphones 104, the wearable sensor device 106, and/or the bedside unit 108.

The headphones 104, the wearable sensor device 106, the bedside unit 108, and the smartphone 102 may each include any wired or wireless communication means suitable for use with any other device 102-108 disposed in the sleeping environment, such as WiFi, Bluetooth, Near Field Communications (NFC), USB, micro USB, or any suitable wired or wireless communications technologies known to one of ordinary skill in the art. For example, the headphones 104 may comprise one or more speakers while the bedside unit 108 comprises one or more biosensors in communication with the one or more speakers of the headphones 104. Furthermore, the audio system 100 may include one or more of the devices 102-108, and is not required to include each device 102-108 shown. Thus, each device 102-108 in the audio system 100 may be optionally included, and only one device 102-108 is needed to determine whether a user is stressing or relaxing, and to dynamically adjust the guided stimulus of the breathing entrainment.

The devices 102-108 of the audio system 100, either alone or in combination, are configured to: select a first target breathing period, a breathing period being an amount of time from a beginning of one inhale to a beginning of a next inhale, output a guiding stimulus to a user, the guiding stimulus being configured to alter a current breathing period of the user towards the first target breathing period over an interval of time at a linear or a non-linear prescribed rate, determine one or more relaxation biometrics of the user that indicate whether the user is relaxing or stressing, analyze the one or more biometrics to determine whether the user is stressing or relaxing, and based at least in part on the one or more determined relaxation biometrics of the user, adjusting at least one of: the guiding stimulus, and the first target breathing period, the first target breathing period being adjusted to a second target breathing period.

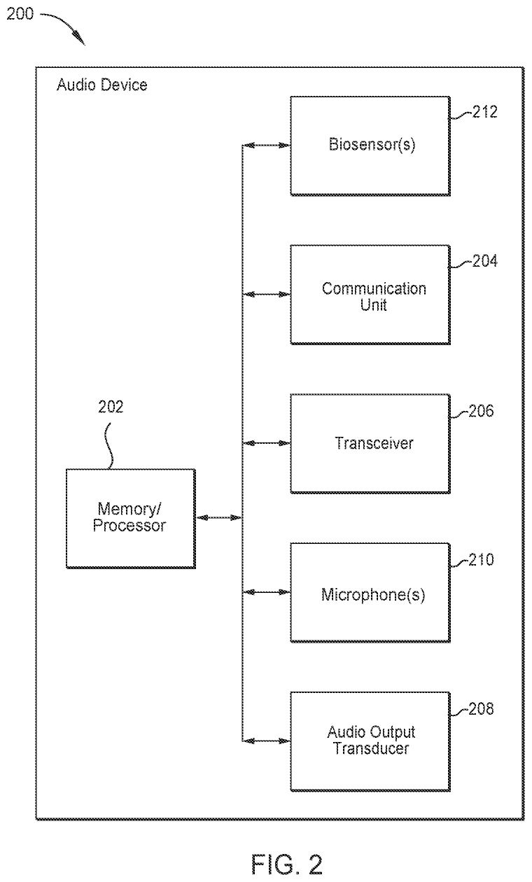

FIG. 2 illustrates example components of an audio device 200, in accordance with certain aspects of the present disclosure. According to an example, the audio device 200 is a wireless wearable audio device. The audio device 200 may be used in an audio system, such as the audio system 100 of FIG. 1. For instance, the audio device 200 may be any device 102-108 in the audio system 100 of FIG. 1. In one example, the audio device 200 is the headphones 104 of FIG. 1. In another example, the audio device 200 is the bedside unit 108 of FIG. 1. The audio device 200 may be used to output and adjust a guiding stimulus along a linear or a non-linear prescribed rate and to determine one or more relaxation biometrics of the user that indicate whether the user is stressing or relaxing.

The audio device 200 includes a memory and processor (or processing unit) 202, communication unit 204, a transceiver 206, a biosensor 212, and an audio output transducer or speaker 208. The memory may include Read Only Memory (ROM), a Random Access Memory (RAM), and/or a flash ROM. The memory stores program code for controlling the memory and processor 202. The memory and processor 202 control the operations of the audio device 200. Any or all of the components in FIG. 2 may be combined into multi-function components.

The processor 202 controls the general operation of the audio device 200. For example, the processor 202 performs process and control for audio and/or data communication. The processor 202 is configured to measure, receive, calculate, or detect at least one biosignal parameter of the subject. In combination with the audio output transducer 208, the processor 202 is configured to output the guiding stimulus. The processor 202 is further configured to analyze the one or more relaxation biometrics, to determine whether a user is relaxing or stressing, and to alter the guiding stimulus. The processor 202 may be further configured to receive input from a subject or user, such as input regarding an initial breath rate per minute and a final breath rate per minute. In at least one example, the processor 202 is disposed on another device in an audio system, such as a smartphone, and is in communication with the audio device 200.

The communication unit 204 facilitates a wireless connection with one or more other wireless devices, such as with other devices in an audio system. For example, the communication unit 204 may include one or more wireless protocol engines such as a Bluetooth engine. While Bluetooth is used as an example protocol, other communication protocols may also be used. Some examples include Bluetooth Low Energy (BLE), NFC, IEEE 802.11, WiFi, or other local area network (LAN) or personal area network (PAN) protocols. The audio device 200 may receive audio files wirelessly via the communication unit 204. Additionally or alternatively, the communication unit 204 may receive information associated with a subject's biosignal parameters, obtained via a contactless sensor. Examples of contactless sensors include a RF sensor, a radar sensor, or an under-bed accelerometer.

The transceiver 206 transmits and receives information via one or more antennae to exchange information with one or more other wireless devices. The transceiver 206 may be used to communicate with other devices in an audio system, such as a bedside unit, a smartphone, and/or a smartwatch. The transceiver 206 is not necessarily a distinct component.

The audio device 200 includes the audio output transducer 208, which may be also known as a driver or speaker. In some examples, more than one output transducer 208 is used. The transducer 208 (that may be part of a microphone) converts electrical signals into sound and converts sound into electrical signals. The transducer 208 is configured to output a guiding stimulus to a user or subject. The transducer 208 outputs audio signals, including adjusted audio signals in an effort to regulate a subject or user's breathing. For example, the transducer 208 may be configured to adjust audio signals in response to a subject's biosignal parameters. In at least one example, the transducer 208 is disposed on another device in an audio system, such as a bedside unit, and is in communication with the audio device 200.

The audio device 200 optionally includes one or more microphones 210. In an aspect, the microphones 210 are used to convert noises into electrical signals. In at least one example, one or more microphones 210 are disposed on another device in an audio system, such as a bedside unit, and are in communication with the audio device 200. The microphone 210 may be used to approximate or measure a user's breath rate per minute.

The audio device 200 includes one or more biosensors 212 used to determine, sense, measure, monitor, or calculate a biosignal parameter of a subject wearing the audio device 200. For example, the one or more biosensors 212 may include a heart rate sensor, a wearable accelerometer, a wearable gyroscope, a wearable sweat sensor, a chest-strap, a RF sensor, a radar sensor, an under-bed accelerometer, an under-bed gyroscope, a motion sensor, among others. The one or more biosensors 212 are configured to determine one or more relaxation biometrics of a user that indicate whether the user is relaxing or stressing. For example, the one or more relaxation biometrics may include heart rate, heart rate variability, EDA, EMG, respiration, perspiration, and blood pressure, among others.

According to an aspect when the audio device 200 is headphones, only one earpiece (ear tip, ear cup) of the audio device 200 includes the biosensor 212. In an aspect, neither earpiece includes a biosensor 212. Instead, a biosensor not on the audio device 200, may remotely detect a biosignal parameter of the subject. In an example, the biosensor 212 detects a subject's heart rate or heart rate variability (HRV) with a sensor disposed on the wrist, such as by utilizing a smartwatch. In an example, the biosensor 212 may be a contactless biosensor. The contactless biosensor is configured to report detected biosignal parameters to the processor 202, for example, via the communication unit 204. In at least one example, the biosensor 212 is disposed on another device in an audio system, such as a smartwatch, and is in communication with the audio device 200.

FIG. 2 illustrates communication between certain modules of an example audio device 200; however, aspects of the disclosure are not limited to the specific illustrated example. According to aspects, any module 202-212 is configured to communicate with any other module in the audio device 200. In one example, all modules 202-212 are connected to and communicate with each other.

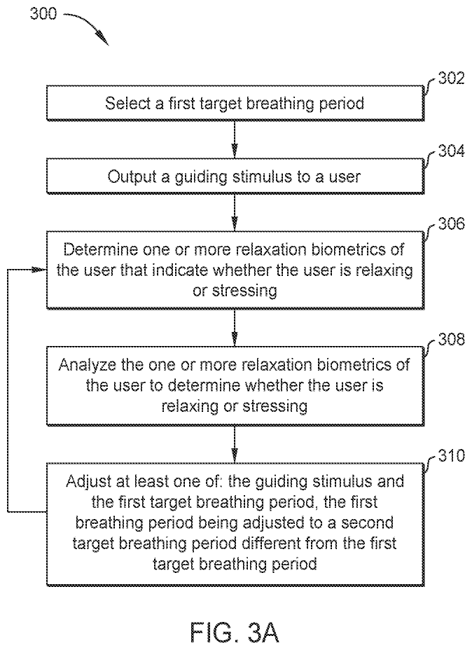

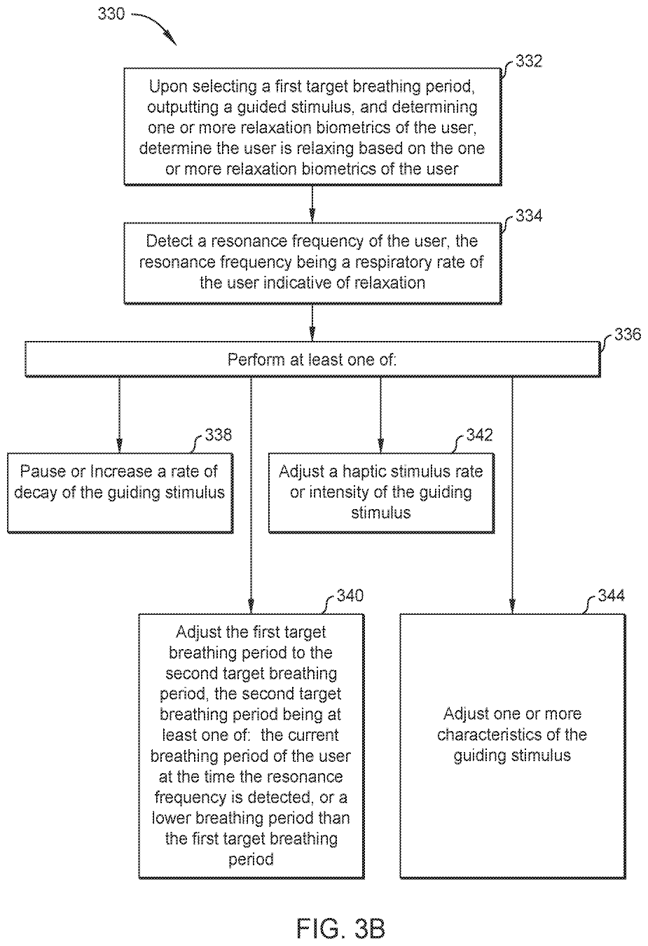

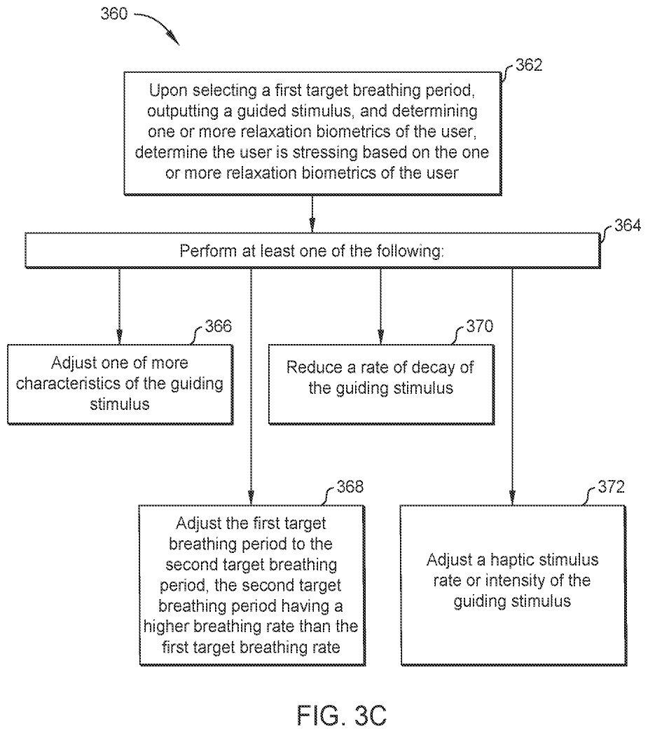

FIG. 3A illustrates an example Method 300 for dynamically adjusting breathing entrainment based on a user's state of relaxation, according to one embodiment. FIG. 3B illustrates an example Method 330 of dynamically adjusting breathing entrainment when the user is determined to be relaxing. FIG. 3C illustrates an example Method 360 of dynamically adjusting breathing entrainment when the user is determined to be stressing. Methods 300, 330, 360 may be implemented utilizing the audio system 100 of FIG. 1 and/or the audio device 200 of FIG. 2. Methods 300, 330, 360, or aspects of Methods 300, 330, 360, may be used in combination with one another.

In 302 of Method 300 of FIG. 3A, a first target breathing period is selected. A breathing period is an amount of time from a beginning of one inhale to a beginning of a next inhale. A first target breathing period is the desired breathing period after the breathing entrainment has been completed. The first target breathing period may be selected by a user, or may be preset or predetermined. An example of a first target breathing period is about a 10 second breathing period or about 6 breaths per minute.

In 304, a guiding stimulus is output. The guiding stimulus is configured to alter a current breathing period of the user towards the first target breathing period over an interval of time, typically at a non-linear prescribed rate. The guiding stimulus may be a pre-produced sound or pre-produced soundtrack. In other embodiments, the guiding stimulus may be one or more of an audio output, a haptic output, a visual output, and a thermal output. The current breathing period of a user is measured using a biometric sensor, such as the biosensor 212 of FIG. 2. The current breathing period may further be measured or approximated using a microphone, such as the microphone 210 of FIG. 2. The current breathing period is generally higher than the first target breathing period, such as about a 6 second breathing period or about 10 breaths per minute. In one embodiment, the current breathing period is measured when method 300 begins to help select the first target breathing period. The current breathing period may be measured one or more times during the entrainment sequence. In some embodiments, the guided stimulus alters the current breathing period of the user towards the first target breathing period over an interval of time in at a linear rate.

In embodiments where the non-linear prescribed rate is utilized, the non-linear prescribed rate is a predetermined breathing entrainment rate or sequence that starts at the current breathing period of the user and ends at the first target breathing period. In other words, the non-linear prescribed rate extends from the current breathing period to the first target breathing period over a total time of entrainment. The total time of entrainment is the amount of time the breathing entrainment sequence or exercise lasts. The non-linear prescribed breathing rate may be a non-linear decay. The non-linear prescribed rate may not follow a step-wise or linear function (e.g., decreasing by one breath per minute). The non-linear prescribed rate may be governed by the inverse of the breathing period, as shown by Equation 1:

$${Rate}_{Breath} = \frac{60 seconds}{Period} \qquad Equation 1$$

In 306, one or more relaxation biometrics of the user are determined. The one or more relaxation biometrics indicate whether the user is relaxing or stressing while using the breathing entrainment. The one or more relaxation biometrics of a user are measured using a biometric sensor, such as the biosensor 212 of FIG. 2. For example, the one or more biosensors 212 may include a heart rate sensor, a wearable accelerometer, a wearable gyroscope, a wearable sweat sensor, a chest-strap, a RF sensor, a radar sensor, an under-bed accelerometer, an under-bed gyroscope, a motion sensor, among others. The one or more biosensors 212 are configured to determine one or more relaxation biometrics of a user that indicate whether the user is relaxing or stressing. For example, the one or more relaxation biometrics may include heart rate, heart rate variability, EDA, EMG, respiration, perspiration, and blood pressure, among others. As noted above, the one or more biometrics may come from a number of devices in an environment.

In 308, the one or more biometrics of the user are analyzed to determine whether the user is stressing or relaxing. For example, a high blood pressure, a high heart rate, or the user's respiratory rate may indicate that the user is stressing, and thus, the breathing entrainment is not optimized for the user. Similarly, a low blood pressure, a low heart rate, or the user's respiratory rate may indicate that the user is relaxing, suggesting that the breathing entrainment and/or output guided stimulus is working as intended for the user. The respiratory rate at which the user's relaxation is detected is the user's resonance frequency. While only one relaxation biometric is needed to determine whether a user is stressing or relaxing, analyzing more than one relaxation biometric may improve the stress/relaxation determination.

In 310, at least one of the guiding stimulus and the first target breathing period are adjusted based on the determination of whether the user is stressing or relaxing. The first target breathing period may be adjusted to a second target breathing period different from the first target breathing period. The specifics of the adjustments made to the guiding stimulus and/or first target breathing period vary based on whether the user is stressing or relaxing, and are discussed further below in FIGS. 3B-3C.

If the user partakes in the breathing entrainment over a period of days (e.g., a night-to-night experience), information may be obtained and analyzed to improve future uses of the breathing entrainment (i.e., a learning algorithm may be implemented). For example, if the user is frequently determined to be stressing due to an uncomfortable starting rate or due to the first target breathing rate being too high or too low, the starting rate or the target breathing rate of the breathing entrainment may be adjusted for future uses. Thus, the next time the user partakes in the breathing entrainment, the user will be more likely to relax in a shorter period of time, as the breathing entrainment will be customized to the particular user.

306-310 of Method 300 may restart one or more times until the user has reached either the first or second target breathing period. Once the user reaches the target breathing period, Method 300 may end, and the guiding stimulus may stop being output. Furthermore, the entrainment system may react to non-respiration based feedback received from the biosensor. For example, if the system receives information indicating the user is asleep, the entrainment may be immediately stopped, or may be stopped gradually over time. Sleep-onset-detection and/or loss-or-awareness algorithm(s) may be utilized to ensure relaxation has occurred, further building confidence in a sleep likelihood of the user.

FIG. 3B illustrates an example Method 330 of dynamically adjusting breathing entrainment when the user is determined to be relaxing. In 332, upon selecting the first target breathing period, outputting the guided stimulus, and determining the one or more relaxation biometrics of the user, the determination is made based on the one or more relaxation biometrics that the user is relaxing.

In 334, a resonance frequency of the user is detected. The user's resonance frequency is the respiratory rate at which the user's relaxation is detected. The user's resonance frequency may be determined through tracking over a period of time. For example, each time the user partakes in the breathing entrainment, or at least the first few times the user partakes in the breathing entrainment, a resonance frequency may be identified and compared to previously identified resonance frequencies. An average of the previously identified resonance frequencies may be used at the resonance frequency for future uses. The resonance frequency may be continually or periodically updated based on new biometric measurements, feedback from the user, or other circumstances. The resonance frequency may further be based on a built model of the user's preferences for achieving the goal of relaxation. In another example, a rate of decay of the guided stimulus may be increased while a relaxation metric of the user (i.e., a peak of the one or more relaxation biometrics) is increasing until the relaxation metric starts to plateau or decrease.

In 336, a determination is made to perform at least one of the actions of 338, 340, 342, and/or 344. One or more of the actions of 338, 340, 342, and/or 344 may be performed either individually or simultaneously. Additionally, one or more of the actions of 338, 340, 342, and/or 344 may be performed one or more times. Moreover, if a first action 338, 340, 342, or 344 is taken and the user shows no improvements or becomes less relaxed, one or more second actions 338, 340, 342, or 344 may be taken. In such an embodiment, the first action taken may be undone. The determination of which action 338, 340, 342, and/or 344 to be performed may be based on the measured relaxation biometrics, factory settings, user input, or a model of the user's preferences for achieving the goal of relaxation.

In 338, a rate of decay of the guided stimulus is paused or increased. In other words, a breathing rate of the user is paused or decreased such that the breathing period remains the same or decreases (e.g., 7 breaths per minute decreased to 6.5 breaths per minute). In some embodiments, the rate of decay of the guided stimulus is paused or increased in response to the resonance frequency being detected. The pausing of the rate of decay of the guided stimulus may be temporary. In another embodiment, the rate of decay may be increased while the relaxation biometric is increasing until the relaxation metric starts to plateau or decrease. In such an embodiment, the rate of decay may then be paused. Adjusting the rate of decay of the guided stimulus may alter the pace and/or time of the breathing entrainment. For example, the breathing entrainment may be shortened, or the linear or non-linear prescribed rate of the guided stimulus may be altered to be steeper or more severe for the remainder of the time.

In 340, the first target breathing period is adjusted to a second target breathing period. In some embodiments, the first target breathing period is adjusted to a second target breathing period in response to the resonance frequency being detected. The second target breathing period is at least one of: the current breathing period of the user at the time the resonance frequency is detected, or a lower breathing period than the first target breathing period. For example, if the first target breathing period is about 6 breaths per minute and the resonance frequency of the user is detected at a breathing period of about 5.8 breaths per minute, the first target breathing period may be adjusted to a second target breathing period of 5.8 breaths per minute or lower, such as 5.5 breaths per minute.

Adjusting the first target breathing period to a second target breathing period may alter the pace and/or time of the breathing entrainment. For example, the breathing entrainment may be shortened, or the linear or non-linear prescribed rate of the guided stimulus may be altered to be steeper or more severe for the remainder of the time. By adjusting the first target breathing period to a second target breathing period, the guided stimulus can become the intended ‘target’ state, where to focus is less on following and more on relaxation. Adjusting the rate of decay of the guided stimulus may alter the pace and/or time of the breathing entrainment.

In 342, a haptic stimulus rate or intensity of the guiding stimulus is adjusted. For example, the haptic stimulus rate may be paused or decreased to result in a decreased breathing rate of the user. In another example, an intensity of the haptic stimulus may be reduced. In one embodiment, the haptic stimulus may slowly face away or decrease entirely. In such an embodiment, the haptic stimulus may switch to audio output.

In 344, one or more characteristics of the guided stimulus are adjusted. For example, the audio output may be adjusted to sounds of higher complexity or to sounds of lower soothing quality. In another example, a density of the audio output, a prevalence of explicit audio cues, or a volume of the audio output may each be increased. In one embodiment, the audio output may slowly face away or decrease entirely. In such an embodiment, the audio output may switch to a haptic stimulus.

In another embodiment, characteristics of a visual output and/or thermal stimulus of the guided stimulus may be altered, such as the guided stimulus being adjusted to mimic sunlight, a rainstorm, or a partly cloudy day, both in light and thermal radiation intensity. For example, characteristics of the visual output and thermal output may be adjusted to reproduce clouds temporarily blocking the sun, resulting in the visual output darkening and the thermal output cooling. In such an embodiment, audio output may be simultaneously adjusted, such as to reproduce a particular time of day, such as birds chirping at dawn or crickets chirping at night. The reproduced feeling of clouds blocking the sun or birds chirping, for example, may be output along the linear or non-linear prescribed rate. In yet another embodiment, the guided stimulus may be a thermal stimulus, such as a heat transducer or a Peltier cooler, guiding a user's breathing by adjusting output temperatures along the linear or non-linear prescribed rate.

Method 330 may restart one or more times until the user has reached either the first or second target breathing period. Furthermore, the entrainment system may react to non-respiration based feedback received from the biosensor. For example, if the system receives information indicating the user is asleep, the entrainment may be immediately stopped, or may be stopped gradually over time. Sleep-onset-detection and/or loss-or-awareness algorithm(s) may be utilized to ensure relaxation has occurred, further building confidence in a sleep likelihood of the user.

FIG. 3C illustrates an example Method 360 of dynamically adjusting breathing entrainment when the user is determined to be stressing. In 362, upon selecting the first target breathing period, outputting the guided stimulus, and determining the one or more relaxation biometrics of the user, the determination is made based on the one or more relaxation biometrics that the user is stressing.

In 364, a determination is made to perform at least one of the actions of 366, 368, 370, and/or 372. One or more of the actions of 366, 368, 370, and/or 372 may be performed either individually or simultaneously. Additionally, one or more of the actions of 366, 368, 370, and/or 372 may be performed one or more times. Moreover, if a first action 366, 368, 370, or 372 is taken and the user shows no improvements or becomes more stressed, one or more second actions 366, 368, 370, or 372 may be taken. In such an embodiment, the first action taken may be undone.

The determination of which action 366, 368, 370, and/or 372 to be performed may be based on the measured relaxation biometrics, factory settings, user input, or a model of the user's preferences for achieving the goal of relaxation. For example, if the user partakes in the breathing entrainment over a period of days, certain sounds may be tracked to determine whether the sounds cause the user stress. Similarly, certain sounds may be tracked to determine whether the sounds cause a population of users stress (e.g., a population of users living geographically near a train or an airport). If certain sounds are known to cause the user or population of users stress, the selection of the action 366, 368, 370, and/or 372 be performed may be in response to the sound being detected or prior to the sound occurring, if such sounds occur around the same time each day.

In 366, one or more characteristics of the guided stimulus are adjusted. The audio cues of the guided stimulus may have a negative valence, thus causing the user to stress. Examples of characteristics that may be adjusted include: a density of the audio output being decreased, a prevalence of explicit audio cues being decreased, a volume of the audio output being decreased, or the audio output being adjusted to sounds of lower complexity (e.g., less layers being mixed) or to sounds of higher soothing quality. In some embodiments, the audio output may fade away or decreases in perceptive loudness to minimize any prolonged stress. In such an embodiment, the audio output may switch to a haptic stimulus.

In another embodiment, characteristics of a visual output and/or thermal stimulus of the guided stimulus may be altered, such as the guided stimulus being adjusted to mimic sunlight, a rainstorm, or a partly cloudy day, both in light and thermal radiation intensity. For example, characteristics of the visual output and thermal output may be adjusted to reproduce lightning during a storm, resulting in the visual output brightening and the thermal output cooling. In such an embodiment, audio output may be simultaneously adjusted, such as to reproduce the sound of thunder and rain. The audio output may further be adjusted to reproduce a particular time of day, such as birds chirping at dawn or crickets chirping at night. The reproduced feeling of thunder and lightning during a storm, for example, may be output along the linear or non-linear prescribed rate. In yet another embodiment, the guided stimulus may be a thermal stimulus, such as a heat transducer or a Peltier cooler, guiding a user's breathing by adjusting output temperatures along the linear or non-linear prescribed rate.

In 368, the first target breathing period is adjusted to a second target breathing period. The second target breathing period is a higher breathing rate than the first target breathing rate. For example, if the first target breathing period is about 6 breaths per minute, the first target breathing period may be adjusted to a second target breathing period of 6.5 breaths per minute. Adjusting the first target breathing period to a second target breathing period may alter the pace and/or time of the breathing entrainment. For example, the breathing entrainment may be extended, or the linear or non-linear prescribed rate of the guided stimulus may be altered to be gentler or less severe for the remainder of the time. Moreover, if the user partakes in the breathing entrainment over a period of days (e.g., a night-to-night experience) and repeatedly becomes stressed at slower breathing rates, the first target breathing rate may be permanently changed to a second target breathing rate known to be more comfortable for the user.

In 370, a rate of decay of the guided stimulus is reduced. If the user is determined to be stressing in the beginning part of the breathing entrainment (e.g., a respiratory rate above 8 breaths per minute), the rate of decay of the guided stimulus may be progressing too quickly for the user, or the starting rate may be uncomfortable, causing the user to stress. In response, the linear or non-linear prescribed rate of the guided stimulus may be reduced such that the rate is altered to be gentler or less severe, or the breathing period may be increased or paused. For example, the breathing rate of 8.5 breaths per minute may be increased to 9 breaths per minute.

If the user is determined to be stressing near the slower part of the breathing entrainment (e.g., a respiratory rate of 8 breaths per minute or below), the user may be having difficulty breathing that slowly. In response, the linear or non-linear prescribed rate of the guided stimulus may be inclined or raised back to a previous level or rate before the stress was detected. Adjusting the rate of decay of the guided stimulus may alter the pace and/or time of the breathing entrainment. For example, the breathing entrainment may be extended, or the linear or non-linear prescribed rate of the guided stimulus may be altered to be gentler or less severe for the remainder of the time. Moreover, if the user partakes in the breathing entrainment over a period of days (e.g., a night-to-night experience), and the user repeatedly becomes stressed at slower breathing rates, the linear or non-linear prescribed rate of the guided stimulus may be permanently reduced, or the linear or non-linear prescribed rate may gradually increase over a period of uses or experiences.

In 372, a haptic stimulus rate or intensity of the guided stimulus is adjusted. For example, the haptic stimulus rate may be paused or decreased to result in a decreased breathing rate of the user. In another example, an intensity of the haptic stimulus may be reduced. In yet another example, audio cues may be substituted for the haptic stimulus. In such an embodiment, the haptic stimulus may slowly fade away and switch to audio output.

As discussed above, if the user partakes in the breathing entrainment over a period of days (e.g., a night-to-night experience), certain sounds or other external sources may be tracked to determine whether the sounds or external sources cause the user stress. Similarly, certain sounds or other external sources may be tracked to determine whether the sounds or external sources cause a population of users stress. If certain sounds or external sources are determined to cause the user or population of users stress, the selection of the action 366, 368, 370, and/or 372 be performed may be in response to the sound being detected or prior to the sound occurring, if such sounds occur around the same time each day. Moreover, if the user partakes in the breathing entrainment over a period of days, and the user repeatedly becomes stressed at slower breathing rates, deep breathing techniques and training may be recommended to the user to assist with future uses of the breathing entrainment.

Method 360 may restart one or more times until the user has reached either the first or second target breathing period. Once the user reaches the target breathing period, Method 360 may end, and the guiding stimulus may stop being output. Furthermore, the entrainment system may react to non-respiration based feedback received from the biosensor. For example, if the system receives information indicating the user is asleep, the entrainment may be immediately stopped, or may be stopped gradually over time. Sleep-onset-detection and/or loss-or-awareness algorithm(s) may be utilized to ensure relaxation has occurred, further building confidence in a sleep likelihood of the user.

Furthermore, aspects of Methods 300, 330, and 360 may be used together. If a user is first determined to be stressing and Method 360 is implemented, upon determining the user is relaxing, one or more of the actions 338, 340, 342, and/or 344 of Method 330 may be implemented. Similarly, if a user is first determined to be relaxing and Method 330 is implemented, upon determining the user is stressing, one or more of the actions 366, 368, 370, and/or 372 of Method 360 may be implemented. Moreover, in each of Methods 300, 330, and 360, the entrainment system may track how often the linear or non-linear prescribed rate is adjusted. Long-term tracking of the adjustments may enable an adaptive entrainment system that modulates its reactive parameters after going through an entrainment sequence.

By analyzing one or more relaxation biometrics and determining whether a user is stressing or relaxing, the breathing entrainment can be dynamically adjusted to help the user relax in the quickest and optimal manner. Moreover, the breathing entrainment can be customized and personalized based on the needs and characteristics of specific users, resulting in a more comfortable breathing entrainment experience. As such, users are more likely to complete the breathing entrainment sequence, and may complete the breathing entrainment in a more effective and easier manner.

Aspects of the present disclosure provide methods, apparatuses, and systems for dynamically adjusting a guided stimulus of a breathing entrainment based on whether a user is stressing or relaxing. According to aspects, the audio device or system described herein is also configured to non-linearly alter a guiding stimulus with a non-linear breath rate per minute sequence to align with a final breathing period, as described in U.S. Patent Application Ser. No. 62/789,343 entitled “Non-Linear Breath Entrainment,” filed on Jan. 7, 2019 (Docket No. WL-18-044-USL), which is hereby incorporated by reference in its entirety.

In the preceding, reference is made to aspects presented in this disclosure. However, the scope of the present disclosure is not limited to specific described aspects. Aspects of the present disclosure may take the form of an entirely hardware embodiment, an entirely software embodiment (including firmware, resident software, micro-code, etc.) or an embodiment combining software and hardware aspects that may all generally be referred to herein as a “component,” “circuit,” “module” or “system.” Furthermore, aspects of the present disclosure may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples of a computer readable storage medium include: an electrical connection having one or more wires, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the current context, a computer readable storage medium may be any tangible medium that can contain, or store a program.

The flowchart and block diagrams in the figures illustrate the architecture, functionality and operation of possible implementations of systems, methods and computer program products according to various aspects. In this regard, each block in the flowchart or block diagrams may represent a module, segment or portion of code, which comprises one or more executable instructions for implementing the specified logical function(s). In some implementations the functions noted in the block may occur out of the order noted in the figures. For example, two blocks shown in succession may, in fact, be executed substantially concurrently, or the blocks may sometimes be executed in the reverse order, depending upon the functionality involved. Each block of the block diagrams and/or flowchart illustrations, and combinations of blocks in the block diagrams and/or flowchart illustrations can be implemented by special-purpose hardware-based systems that perform the specified functions or acts, or combinations of special purpose hardware and computer instructions.

1. A method for breathing entrainment, comprising:
selecting a first target breathing period, a breathing period being an amount of time from a beginning of one inhale to a beginning of a next inhale;
outputting a guiding stimulus to a user, the guiding stimulus being configured to alter a current breathing period of the user towards the first target breathing period over an interval of time at a non-linear prescribed rate;
determining one or more relaxation biometrics of the user that indicate whether the user is relaxing or stressing; and
based at least in part on the one or more determined relaxation biometrics of the user, adjusting at least one of:
the guiding stimulus; and
the first target breathing period, the first target breathing period being adjusted to a second target breathing period.

2. The method of claim 1, wherein determining the one or more relaxation biometrics of the user comprises detecting a resonance frequency of the user, the resonance frequency being a respiratory rate of the user indicative of relaxation.

3. The method of claim 2, wherein adjusting at least one of the guiding stimulus and the first target breathing period comprises performing at least one of:
pausing or increasing a rate of decay of the non-linear prescribed rate of the guiding stimulus in response to detecting the resonance frequency of the user;
adjusting a haptic stimulus rate of the guiding stimulus;
adjusting a haptic stimulus intensity of the guiding stimulus;
adjusting an audio output of the guided stimulus to sounds of higher complexity;
adjusting a visual output of the guided stimulus; and
adjusting a thermal output of the guided stimulus.

4. The method of claim 2, wherein adjusting at least one of the guiding stimulus and the first target breathing period comprises adjusting the first target breathing period to the second target breathing period in response to detecting the resonance frequency of the user, the second target breathing period being at least one of:
the current breathing period of the user at the time the resonance frequency is detected; or
a lower breathing period than the first target breathing period.

5. The method of claim 1, wherein determining the one or more relaxation biometrics of the user comprises determining the user is stressed based on the one or more relaxation biometrics of the user, and wherein adjusting at least one of the guiding stimulus and the first target breathing period comprises performing at least one of:
decreasing a volume of an audio output of the guiding stimulus;
adjusting the audio output of the guiding stimulus to sounds of higher soothing quality;
adjusting the audio output of the guiding stimulus to sounds of lower complexity;
adjusting the first target breathing period to the second target breathing period, the second target breathing period having a higher breathing rate than the first target breathing period;
reducing a rate of decay of the non-linear prescribed rate of the guiding stimulus;
reducing a haptic stimulus intensity of the guiding stimulus;
adjusting a haptic stimulus rate of the guiding stimulus;
adjusting a visual output of the guided stimulus; and
adjusting a thermal output of the guided stimulus.

6. The method of claim 1, wherein the one or more relaxation biometrics are determined using one or more biometric sensors, and wherein the guiding stimulus is at least one of audio output, a haptic stimulus, a visual output, and a thermal output.

7. The method of claim 1, wherein the one or more relaxation biometrics are selected from the group consisting of heart rate, heart rate variability, electrodermal activity, electromyography, respiration, perspiration, and blood pressure.

8. A wearable audio device, comprising:
at least one biosensor for determining one or more relaxation biometrics of a user that indicate whether the user is relaxing or stressing;
at least one speaker configured to output a guiding stimulus, the guiding stimulus being configured to alter a current breathing period of the user towards a first target breathing period over an interval of time at a non-linear prescribed rate, a breathing period being an amount of time from a beginning of one inhale to a beginning of a next inhale; and
a processing unit configured to:
select the first target breathing period; and
based at least in part on the one or more determined relaxation biometrics of the user, adjust at least one of:
the guiding stimulus; and
the first target breathing period, the first target breathing period being adjusted to a second target breathing period.

9. The wearable audio device of claim 8, wherein determining the one or more relaxation biometrics of the user comprises detecting a resonance frequency of the user, the resonance frequency being a respiratory rate of the user indicative of relaxation.

10. The wearable audio device of claim 9, wherein adjusting at least one of the guiding stimulus and the first target breathing period comprises performing at least one of:
pausing or increasing a rate of decay of the non-linear prescribed rate of the guiding stimulus in response to detecting the resonance frequency of the user;
adjusting a haptic stimulus rate of the guiding stimulus;
adjusting a haptic stimulus intensity of the guiding stimulus;
adjusting an audio output of the guided stimulus to sounds of higher complexity;
adjusting a visual output of the guided stimulus; and
adjusting a thermal output of the guided stimulus.

11. The wearable audio device of claim 9, wherein adjusting at least one of the guiding stimulus and the first target breathing period comprises adjusting the first target breathing period to the second target breathing period in response to detecting the resonance frequency of the user, the second target breathing period being at least one of:
the current breathing period of the user at the time the resonance frequency is detected; or
a lower breathing period than the first target breathing period.

12. The wearable audio device of claim 8, wherein determining the one or more relaxation biometrics of the user comprises determining the user is stressed based on the one or more relaxation biometrics of the user, and wherein adjusting at least one of the guiding stimulus and the first target breathing period comprises performing at least one of:
decreasing a volume of an audio output of the guiding stimulus;
adjusting the audio output of the guiding stimulus to sounds of higher soothing quality;
adjusting the audio output of the guiding stimulus to sounds of lower complexity;
adjusting the first target breathing period to the second target breathing period, the second target breathing period having a higher breathing rate than the first target breathing period;
reducing a rate of decay of the non-linear prescribed rate of the guiding stimulus;
reducing a haptic stimulus intensity of the guiding stimulus;
adjusting a haptic stimulus rate of the guiding stimulus;
adjusting a visual output of the guided stimulus; and
adjusting a thermal output of the guided stimulus.

13. The wearable audio device of claim 8, wherein the one or more relaxation biometrics are determined using one or more biometric sensors, and wherein the guiding stimulus is at least one of audio output, a haptic stimulus, a visual output, and a thermal output.

14. The wearable audio device of claim 8, wherein the one or more relaxation biometrics are selected from the group consisting of heart rate, heart rate variability, electrodermal activity, electromyography, respiration, perspiration, and blood pressure.

15. An audio system, comprising:
at least one biosensor for determining one or more relaxation biometrics of a user that indicate whether the user is relaxing or stressing;
at least one speaker configured to output a guiding stimulus, the guiding stimulus being configured to alter a current breathing period of the user towards a first target breathing period over an interval of time at a non-linear prescribed rate, a breathing period being an amount of time from a beginning of one inhale to a beginning of a next inhale; and
a processing unit configured to:
select the first target breathing period; and
based at least in part on the one or more determined relaxation biometrics of the user, adjust at least one of:
the guiding stimulus; and
the first target breathing period, the first target breathing period being adjusted to a second target breathing period.

16. The audio system of claim 15, wherein determining the one or more relaxation biometrics of the user comprises detecting a resonance frequency of the user, the resonance frequency being a respiratory rate of the user indicative of relaxation.

17. The audio system of claim 16, wherein adjusting at least one of the guiding stimulus and the first target breathing period comprises performing at least one of:
pausing or increasing a rate of decay of the non-linear prescribed rate of the guiding stimulus in response to detecting the resonance frequency of the user;
adjusting a haptic stimulus rate of the guiding stimulus;
adjusting a haptic stimulus intensity of the guiding stimulus;
adjusting an audio output of the guided stimulus to sounds of higher complexity;
adjusting a visual output of the guided stimulus; and
adjusting a thermal output of the guided stimulus.

18. The audio system of claim 16, wherein adjusting at least one of the guiding stimulus and the first target breathing period comprises adjusting the first target breathing period to the second target breathing period in response to detecting the resonance frequency of the user, the second target breathing period being at least one of:
the current breathing period of the user at the time the resonance frequency is detected; or
a lower breathing period than the first target breathing period.

19. The audio system of claim 15, wherein determining the one or more relaxation biometrics of the user comprises determining the user is stressed based on the one or more relaxation biometrics of the user, and wherein adjusting at least one of the guiding stimulus and the first target breathing period comprises performing at least one of:
decreasing a volume of an audio output of the guiding stimulus;
adjusting the audio output of the guiding stimulus to sounds of higher soothing quality;
adjusting the audio output of the guiding stimulus to sounds of lower complexity;
adjusting the first target breathing period to the second target breathing period, the second target breathing period having a higher breathing rate than the first target breathing period;
reducing a rate of decay of the non-linear prescribed rate of the guiding stimulus;
reducing a haptic stimulus intensity of the guiding stimulus;
adjusting a haptic stimulus rate of the guiding stimulus;
adjusting a visual output of the guided stimulus; and
adjusting a thermal output of the guided stimulus.

20. The audio system of claim 15, wherein the one or more relaxation biometrics are determined using one or more biometric sensors, wherein the guiding stimulus is at least one of audio output, a haptic stimulus, a visual output, and a thermal output, and wherein the one or more relaxation biometrics are selected from the group consisting of heart rate, heart rate variability, electrodermal activity, electromyography, respiration, perspiration, and blood pressure.
